(19) United States
(12) Patent Application Publication
(10) Pub. No.: US 2004/0267374 A1
(43) Pub. Date: Dec. 30, 2004

(54) Hip joint endoprosthesis

(76) Inventor: Arno Friedrichs, Kulmbach (DE)

Correspondence Address: WILLIAM COLLARD, COLLARD & ROE, P.C., 1077 NORTHERN BOULEVARD, ROSLYN, NY, 11576, US

(21) Appl. No.: 10/874,055

(22) Filed: Jun. 22, 2004

(30) Foreign Application Priority Data
Jun. 27, 2003 (DE) 103 29 076.1

(51) Int. Cl. A61F 2/32

(52) U.S. Cl. 623/22.15; 623/22.3; 623/23.17; 623/23.41

(57) ABSTRACT

A hip joint endoprosthesis which comprises a shank, a joint ball at the upper end region of the shank, a joint socket, within which the joint ball is mounted to be rotationally movable, and a joint gap present between the joint ball and the joint socket. The joint socket and/or the joint ball has in the interior thereof a closed liquid-filled, gas-filled or gel-filled region. This is separated from the joint gap only by a thin, resiliently deformable edge region of the joint socket or joint ball.

### BACKGROUND OF THE INVENTION

1. Field of the Invention

The invention relates to a hip joint endoprosthesis which comprises a shank, a joint ball at the upper end region of the shank, a joint socket in which the joint ball is rotationally movably mounted in the joint socket, and a joint gap present between the joint ball and the joint socket.

2. The Prior Art

Hip joint endoprostheses of that kind are already known. They are used as a substitute for degenerated natural hip joints in the human body. For insertion of the shank with the joint ball at the upper end region thereof, the upper end of the femur is initially cleanly sawn off. Insertion of the downwardly tapering shank into the marrow channel with or without use of bone cement is then carried out. For insertion of the joint socket, the natural joint socket of the human body is routed away precisely and then the artificial joint socket is inserted into the routed-out region. The joint socket can be fixed via a thread on its outer region, which is screwed into the routed-out natural joint socket. Alternatively thereto, the joint socket can also be cemented in place in the routed-out natural joint socket and in a given case additionally fixed with screws.

Known hip joint endoprosthesis generally consist of metallic materials, ceramic materials, plastics materials or a mix of metal and ceramic. A problem of known artificial hip joints is that due to the loads, which occur in daily life, in the joint, friction and wear arise which with time can lead to undesired secondary effects such as notch fissures in the joint ball or the joint socket or even to detaching of the shank from the femur. These undesired effects occur to increased extent with production inaccuracies. The consequences are a reduced service life of the hip joint endoprosthesis.

### SUMMARY OF THE INVENTION

It is an object of the invention to provide a hip joint endoprosthesis in which the frequency of undesired secondary effects is reduced.

This object is achieved by hip joint endoprosthesis which comprises a shank, a joint ball at the upper end region of the shank, a joint socket, within which the joint ball is mounted to be rotationally movable, and a joint gap between the joint ball and the joint socket. The joint socket and/or the joint ball has in the interior thereof a closed liquid-filled, gas-filled or gel-filled region. This is separated from the joint gap only by a thin, resiliently deformable edge region of the joint socket or joint ball.

The advantages of the invention consist particularly in that by virtue of the closed liquid-filled, gas-filled or gel-filled region, in conjunction with the thin, resiliently deformable edge region, the contact area between joint socket and joint ball is increased, in the strongly loaded state of the joint, by resilient deformation of the part of the prosthesis having the closed region. Through the departure from the rigid and hard shapes of the prosthesis parts, which are usual in the state of the art, the hip joint endoprosthesis of the present invention can dynamically adapt to the respective load situation. Regardless of whether the instantaneously occurring load of the joint acts in the direction of the longitudinal axis of the shank, in a direction parallel to the longitudinal axis of the shank or at an angle of, for example ±45° relative to the longitudinal axis of the shank, the load is distributed to a greater region of the contact area between joint ball and joint socket by the resilient deformation which occurs in the part of the prosthesis having the closed liquid-filled, gas-filled or gel-filled region. This corresponds with a reduction in punctiform loads or loads occurring in confined regions. This reduction in the loading of small regions in turn reduces wear and abrasion of joint parts in the vicinity of the joint gap. The consequence is a longer service life of the hip joint endoprosthesis.

The liquid, gas or gel is preferably introduced in a cushion which is impermeable for the respective medium. This has the advantage that when metal is a material of the hip joint endoprosthesis, no corrosion can arise. In addition, this has the advantage that if small cracks occur in the material of the endoprosthesis after long-standing use, no liquid, gas or gel can issue into the part of the human body surrounding the joint.

### BRIEF DESCRIPTION OF THE DRAWINGS

Other objects and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 shows a sketch of a sectional illustration of a hip joint endoprosthesis according to a first embodiment of the invention;

FIG. 2 shows a sketch of a sectional illustration of a hip joint endoprosthesis according to a second embodiment of the invention;

FIG. 3 shows a sketch of a sectional illustration of a hip joint endoprosthesis according to a third embodiment of the invention; and

FIG. 4 shows a sketch of a sectional illustration of a hip joint endoprosthesis according to a fourth embodiment of the invention.

### DETAILED DESCRIPTION OF THE PREFERRED EMBODIMENT

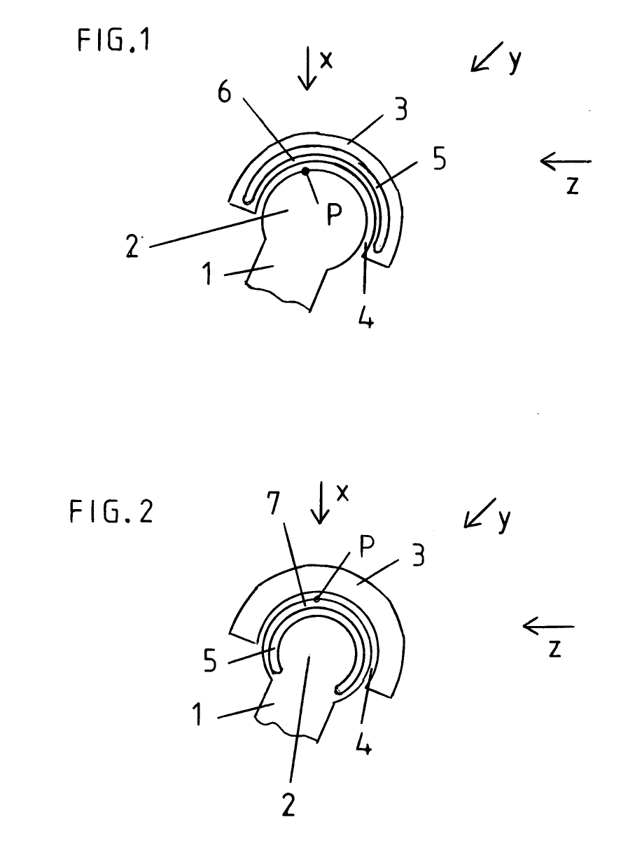

Referring now in detail to the drawings, FIG. 1 shows a sketch of a sectional illustration of a hip joint endoprosthesis according to a first embodiment for the invention. The illustrated hip joint endoprosthesis comprises a shank 1 which is shown cut away and at the upper end region of which is provided a joint ball 2. Joint ball 2 is either a separate component which is placed on the shank or a component integrally formed with the shank.

In addition, the illustrated hip joint endoprosthesis comprises a joint socket 3, within which the joint ball 2 is mounted to be rotationally movable. A joint gap 4 is present between joint ball 2 and joint socket 3. A closed liquid-filled region 5 is provided within joint socket 3. This region is constructed to be of substantially hemispherical shell shape and extends parallel to joint gap 4. Edge region 6, which is disposed between the liquid-filled region 5 and joint gap 4, of joint socket is formed to be thin and resiliently deformable.

If a strong pressure is exerted on the joint socket from above in the direction of arrow x, then in a first brief moment in the region around point P there arises between joint ball 2 and joint socket 3 a strong contact which with constant loading and motion can lead to wear and abrasion. These undesired effects occur only to a significantly reduced extent in the joint illustrated in FIG. 1 since through the strong loading occurring in the first brief moment the thin edge region 6 of joint socket 3 is deformed in the vicinity of point P. Due to this deformation of thin edge region 6, pressure is exerted on the liquid disposed in region 5. This pressure is passed on to parts, which are more remote from point P, of the thin, resiliently deformable edge region 6, which due to this pressure is pressed against joint socket 2 more strongly than in the unloaded state. These processes correspond with a distribution of the loading, which is initially present in a confined region around point P, of the joint to a wider region.

If exertion of pressure in direction x has ended, resiliently deformable edge region 6 again adopts its original form.

This effect of increasing the contact area between the joint ball and joint socket on occurrence of strong loads in order to avoid excessive loading of smaller regions of the joint ball and joint socket is achieved not only when the exerted pressure acts in the direction of the arrow x, but also when the exerted pressure acts in the direction of arrow y or in the direction of arrow z.

FIG. 2 shows a sketch of a sectional illustration of the hip joint endoprosthesis according to a second embodiment of the invention. The illustrated hip joint endoprosthesis comprises a shank 1 which is shown cut away and at the upper end region of which is provided a joint ball 2. This joint ball is either a separate component which is placed on the shank or a component constructed integrally with the shank.

In addition, the illustrated hip joint endoprosthesis comprises a joint socket 3, within which the joint ball 2 is mounted to be rotationally movable. A joint gap 4 is present between joint ball 2 and joint socket 3.

A closed liquid-filled region 5 is provided within joint ball 2. This is formed to be of substantially hemispherical shell shape and extends parallel to the joint gap. Edge region 7, which is disposed between liquid-filled region 5 and joint gap 4, of joint ball 2 is formed to be thin and resiliently deformable.

If a strong pressure is exerted on the joint socket from above in the direction of arrow x, then in a first brief moment in the region around point P a strong contact occurs between joint ball 2 and joint socket 3 with constant loading and the joint in motion can lead to wear and abrasion. These undesired effects occur only to a significantly reduced extent in the joint illustrated in FIG. 2, since due to the strong loading which occurs in the first brief moment, thin edge region 7 of joint ball 2 is deformed in the vicinity of point P. Due to this deformation of thin edge region 7, pressure is exerted on the liquid disposed in region 5. This pressure is passed on to parts, which are more remote from the point P, of the thin, resiliently deformable edge region 7, which due to this pressure are pressed more strongly against joint socket 3 than in the unloaded state. These processes correspond with a distribution of the loading, which is initially present in a confined region around point P, of the joint to a wider region.

If exertion of pressure in direction x has ended, the resiliently deformable edge region 7 again adopts its original shape.

The afore-described effect of increasing the contact area between joint ball and joint socket on occurrence of strong loads in order to avoid excessive loading of small regions of the joint ball and joint socket is achieved not only when the exerted pressure acts in the direction of arrow x, but also when the exerted pressure acts in the direction of arrow y or in the direction of arrow z.

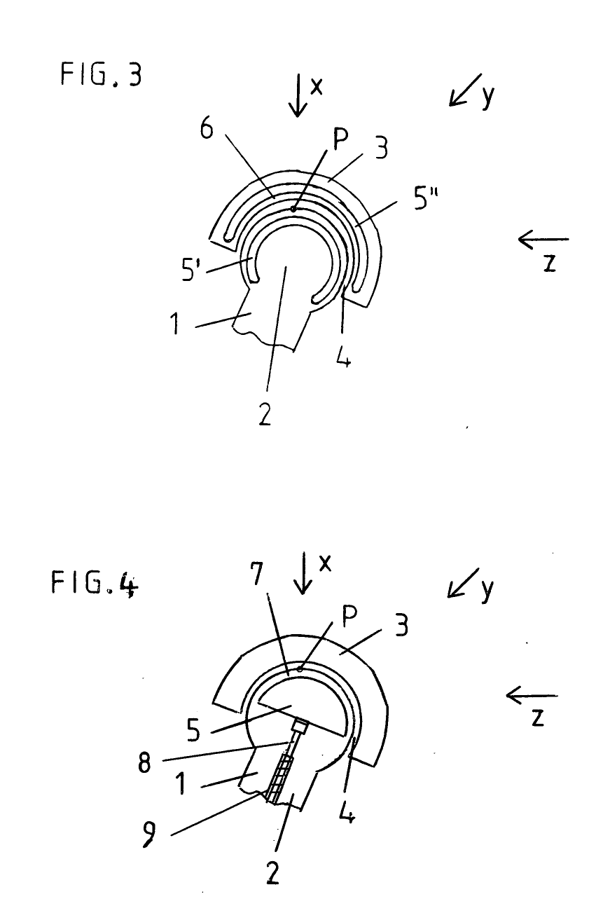

FIG. 3 shows a sketch of a sectional illustration of a hip joint endoprosthesis according to a third embodiment of the invention. The hip joint endoprosthesis comprises a shank 1 which is shown cut away and at the upper end region of which is provided a joint ball 2. This joint ball is either a separate component which is placed on the shank or a component integrally formed with the shank. A closed liquid-filled region 5′ is provided within joint ball 2.

In addition, the illustrated hip joint endoprosthesis comprises a joint socket 3, within which joint ball 2 is mounted to be rotationally movable. A closed, liquid-filled region 5″ is provided within joint socket 3.

A joint gap 4 is present between joint ball 2 and joint socket 3. Edge region 6, which is disposed between liquid-filled region 5″ of joint socket 3 and joint gap 4, of the joint socket is formed to be thin and resiliently deformable. Edge region 7, which is disposed between liquid-filled region 5′ of joint ball 2 and joint gap 4 of joint ball 2 is similarly formed to be thin and resiliently deformable. Regions 5′ and 5″ are each formed to be substantially of hemispherical shell shape and extend parallel to the joint gap.

If a strong force is exerted on the joint socket from above in the direction of arrow x then there occurs in a first brief moment in the region around point P between joint ball 2 and joint socket 3 a strong contact which with constant loading and the joint in motion can lead to wear and abrasion. These undesired effects occur only to significantly reduced extent in the joint illustrated in FIG. 3, since through the strong loading occurring in the first brief moment, thin edge region 6 of the joint socket and thin edge region 7 of the joint ball are deformed in the vicinity of point P. Due to this deformation of thin edge regions 6 and 7, pressure is exerted on the liquids disposed in the regions 5′ and 5″. This pressure is passed to parts, which are more remote from point P, of the thin, resiliently deformable edge regions 6 and 7. These more remote parts of the edge regions are, by virtue of the exerted pressure, pressed more strongly against one another than in the unloaded state. These processes correspond with a distribution of the loading, which is initially present in a confined region around point P, to a wider region.

If exertion of pressure in direction x has ended, then resiliently deformable edge regions 6 and 7 again adopt their original form.

The effect of increasing the contact area between the joint ball and joint socket on occurrence of strong loads in order to avoid excessive loading of small regions of the joint ball and joint socket is achieved not only when the exerted pressure acts in the direction of arrow x, but also when the exerted pressure acts in the direction of arrow y or in the direction of arrow z.

FIG. 4 shows a sketch of a sectional illustration of a hip joint endoprosthesis according to a fourth embodiment of the invention. The illustrated hip joint endoprosthesis comprises a shank 1 which is shown cut away and at the upper end region of which there is provided a joint ball 2. This joint ball is either a separate component which is placed on the shank or a component formed integrally with the shank.

In addition, the illustrated hip joint endoprosthesis comprises a joint socket, within which the joint ball 2 is mounted to be rotationally movable. A joint gap 4 is present between the joint ball 2 and the joint socket 3.

A closed liquid-filled region 5 is provided within joint ball 2. This is formed to be of substantially hemispherical shape and extends parallel to joint gap 4. Edge region 7, which is disposed between liquid-filled region 5 and joint gap 4, of joint ball 2 is formed to be thin and resiliently deformable.

If a strong pressure is exerted on the joint socket from above in the direction of arrow x then in a first brief moment in the region around point P there occurs between joint ball 2 and joint socket 3 a strong contact which with constant loading and the joint in motion can lead to wear and abrasion. These desired effects occur only to a significantly reduced extent in the joint illustrated in FIG. 4, since through the strong loading occurring in the first brief moment thin edge region 7 of joint ball 2 is deformed in the vicinity of the point P. Due to this deformation of the edge region 7, pressure is exerted on the liquid disposed in region 5. This pressure is passed on to parts, which are more remote from the point P, of the thin, resiliently deformable edge region 7, which due to this pressure are pressed more strongly against the joint socket 3 than in the unloaded state. These processes correspond with a distribution of the load, which is initially present in a confined region around point P, of the joint to a wider region.

If exertion of pressure in direction x is ended, then the resiliently deformable edge region 7 again adopts its original form.

The effect of increasing the contact area between the joint ball and joint socket, on occurrence of strong loads, in order to avoid excessive loading of a small region of the joint ball and joint socket is achieved not only when the exerted pressure acts in the direction of arrow x, but also when the exerted pressure acts in the direction of arrow y or in the direction of arrow z.

In addition, the hip joint endoprosthesis shown in FIG. 4 has a ram 8 in shank 1. By means of this ram 8, the pressure acting on closed region 5 can be changed prior to insertion of the endoprosthesis into the human body, in the sense of a pre-adjustment in order to better adapt the pressure conditions, which prevail in the inserted state of the endoprosthesis, to the body weight of the respective person. In order to be able to undertake this pre-adjustment, ram 8 has a thread 9 at its outer surface so that through a screwing movement of the ram, the pressure acting on region 5 can be increased or decreased according to the respective need.

The liquid disposed in the regions 5, 5′ and 5″ is preferably introduced in a liquid-impermeable cushion. This has the advantage that the liquid cannot come into contact with further material of the joint ball or the joint socket. It is thereby ensured that no undesired effects occur, which could be caused by a direct contact between the liquid and the further material. For example, when metal is used as the further material, corrosion is prevented. Moreover, through the use of a liquid-impermeable cushion it is ensured that even if—notwithstanding everything—cracks in the material occur in long-term use, the migration of liquid in the hip joint endoprosthesis into adjacent regions of the human body is avoided.

A further advantageous refinement of the invention consists in using in each of the regions 5, 5′ and 5″ not only a single liquid cushion, but a plurality of liquid cushions. This offers, for example, the possibility of using screws for fixing the artificial joint socket in the routed-out natural joint socket of the human body.

In the above-described embodiments, the starting point was always that the closed region 5, 5′, 5″ is filled with liquid. Alternatively, it is also possible to fill the closed region 5, 5′, 5″ with a gas or a gel. Water or oil, for example, can be used as liquid, and oxygen, nitrogen or liquid gas can be used as gas.

Suitable materials for that component of the hip joint endoprosthesis which has the closed region are, in particular, plastics materials, since plastics materials can be readily managed in production have the resilient deformability necessary for the invention.

Accordingly, while only a few embodiments of the present invention have been shown and described, it is obvious that many changes and modifications may be made thereunto without departing from the spirit and scope of the invention.

What is claimed is:

1. A hip joint endoprosthesis comprising:
a shank;
a joint ball disposed at an upper end region of said shank;
a joint socket, wherein the joint ball is mounted within said joint socket to be rotationally movable; and
a joint gap present between the joint ball and the joint socket,
wherein at least one of the joint socket and the joint ball has in an interior thereof a closed liquid-filled, gas-filled or gel-filled region separated from the joint gap only by a thin, resiliently deformable edge region.

2. The hip joint endoprosthesis according to claim 1, wherein the liquid, gas or gel is disposed in a cushion which is impermeable by the liquid, gas or gel.

3. The hip joint endoprosthesis according to claim 2, wherein the liquid, gas or gel is disposed in a plurality of cushions.

4. The hip joint endoprosthesis according to claim 1, wherein said endoprosthesis comprises an adjustable ram by means of which pressure acting on the closed region is adjustable.

5. The hip joint endoprosthesis according to claim 4, wherein the ram has an external thread, the ram being inserted into the joint socket or joint ball and wherein adjustment of the pressure acting on the closed region can be carried out by a screwing movement of the ram.

6. The hip joint endoprosthesis according to claim 1, wherein the closed region is constructed to be of substantially hemispherical shell shape or hemispherical shape.

7. The hip joint endoprosthesis according to claim 1, wherein the closed region extends parallel to the joint gap.
